(19) United States
(12) Patent Application Publication
(10) Pub. No.: US 2014/0023111 A1
(43) Pub. Date: Jan. 23, 2014

(54) DEVICE FOR MEASURING RELATIVE HUMIDITY AND TEMPERATURE OF THE AIR IN A DIPHASIC MEDIUM

(71) Applicants: Jerome Gahartian, Paris (FR); Julien Tissot, Lemainville (FR)

(72) Inventors: Jerome Gahartian, Paris (FR); Julien Tissot, Lemainville (FR)

(73) Assignee: INSTITUT NATIONAL DE RECHERCHE EN SCIENCES ET TECHNOLOGIES POUR L'ENVIRONNEMEN, Antony Cedex (FR)

(21) Appl. No.: 14/009,643

(22) PCT Filed: Apr. 4, 2012
(86) PCT No.: PCT/FR2012/050726

§ 371 Date: Oct. 3, 2013

(30) Foreign Application Priority Data
Apr. 12, 2011 (FR) 1153170

(51) Int. Cl. G01K 13/00; G01N 33/00

(52) U.S. Cl. CPC G01K 13/00; G01N 33/0004
USPC 374/142

(57) ABSTRACT

A relative humidity sensor 1 and a temperature sensor 2 are placed in a tube 4 traversed by an ascending air current, the speed of ascent of the air being less than the equilibrium speed of the water droplets in an ascending air.

The invention relates to a method and a device for measuring relative humidity and temperature of the air in a diphasic medium, particularly in an air/water medium, and more particularly in an air/water-droplet medium.

In such a medium, for example in the case of atomization of water in air, the temperature of the water and that of the air are different as long as the air has not reached water vapor saturation. When the air and water temperatures are different, a temperature sensor normally indicates the temperature of the air. If it is struck by a water droplet, however, the sensor indicates the temperature of the water droplet rather than that of the air. In the same way, a relative humidity sensor normally indicates the relative humidity of the air. If it is struck by a water droplet, the sensor indicates a relative humidity of 100%, corresponding to that of water.

It is an object of the invention to provide a method and a device for measuring the relative humidity and temperature in a diphasic air/water medium, which is free of the risk of measuring the relative humidity and temperature of the aqueous phase of the medium.

The invention relates to a method for measuring the relative humidity and temperature of the air in a diphasic medium consisting of air and water droplets, by means of a relative humidity sensor and a temperature sensor which are arranged inside a vertical tube, characterized in that the diphasic medium is subjected to an ascending movement in the tube at a speed less than the equilibrium speed of the water droplets in ascending air.

Advantageously, the ascending movement is ensured by suction in the upper part of the tube.

The invention also relates to a device for measuring relative humidity and temperature of the air in a diphasic medium consisting of air and water droplets, in which a relative humidity sensor and a temperature sensor are connected to a measurer, characterized in that it comprises a substantially vertical tube through which an ascending air flow passes, the relative humidity sensor and the temperature sensor being placed inside the tube, and in that the speed of ascent of the air in the tube is less than the equilibrium speed of the water droplets in ascending air.

Advantageously, the air flow is ensured by suction in the upper part of the tube.

Advantageously the suction is ensured by means of a pump.

Advantageously, the average distance between the lower opening of the tube and the relative humidity and temperature sensors is about two times the diameter of the tube.

Other objects, details, characteristics and advantages of the invention will emerge from the following description of a particular embodiment of the invention, which is given by way of illustration and without implying limitation, with reference to the appended drawings.

In the drawings:

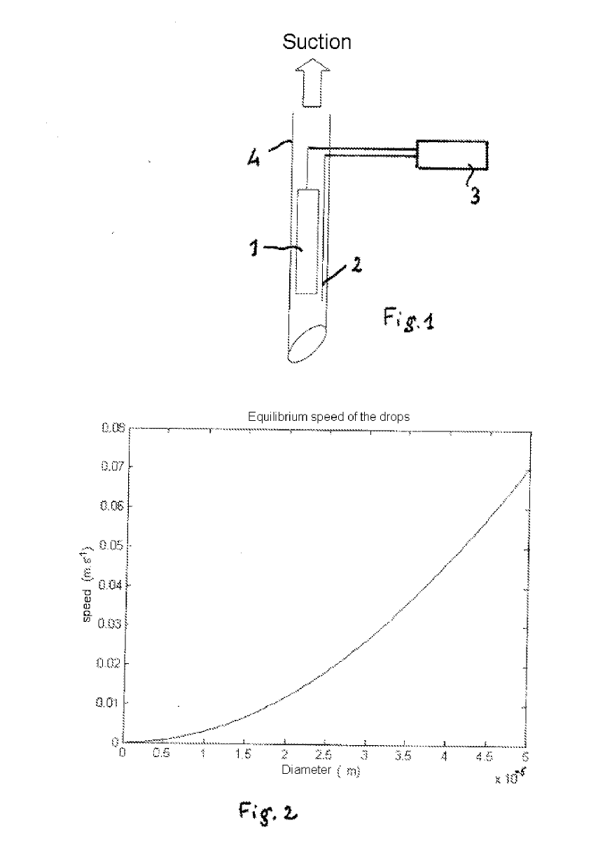

FIG. 1 is a simplified diagram of a device for measuring relative humidity and temperature of the air in a diphasic air/water medium, according to one embodiment of the invention;

FIG. 2 is a diagram representing the equilibrium speed of the water droplets in ascending air, as a function of their diameter.

The device for measuring relative humidity and temperature of the air in a diphasic medium comprises a relative humidity sensor 1 and a temperature sensor 2, which are connected to a measurer 3. According to the invention, in a diphasic air/water medium, the relative humidity sensor 1 and the temperature sensor 2 are placed inside a substantially vertical tube 4 subjected to suction in its upper part in order to cause the air in the tube 4 to ascend. This ascending of air in the tube is ensured by a pump (not shown) at a speed less than the equilibrium speed of the water droplets in ascending air, in order to prevent the water droplets from rising in the tube 4. The result is that only the air flows in the tube. A first consequence is that the measurements of the temperature and relative humidity are not perturbed by the impact of water droplets on the sensors. A second consequence is that the replenishment of the air in the tube makes it possible to measure the average value of the temperature and relative humidity of the air flowing outside the tube 4. The combination of the tube 4 and the pump constitutes a device for protecting the sensors 1, 2 against the impact of the water droplets in suspension in the air.

In FIG. 2, the curve represents the equilibrium speed of the water droplets in ascending air, as a function of the diameter of the water droplets. For water droplets with a given diameter, the speed of the air in the tube must be less than the equilibrium speed: in FIG. 2, the point representing the speed of the air in the tube must lie below the curve.

In an embodiment which is given by way of nonlimiting example, the tube 4 has a diameter of the order of 1 cm and a length of about 10 cm. The average distance between the lower opening of the tube 4 and the relative humidity 1 and temperature 2 sensors is about two times the diameter of the tube 4, that is to say about 2 cm. For an average diameter of the water droplets of 3.10⁻⁵ m, the flow speed of the air in the tube 4 is about 0.02 m·s⁻¹.

According to the invention, an ascending air flow with a speed less than the equilibrium speed of the water droplets in ascending air passes through the tube 4. The air flow is ensured by suction by means of a pump, in the upper part of the tube 4.

1-6. (canceled)

7. A method for measuring the relative humidity and temperature of the air in a diphasic medium consisting of air and water droplets, by means of a relative humidity sensor and a temperature sensor which are arranged inside a vertical tube, wherein the diphasic medium is subjected to an ascending movement in the tube at a speed less than the equilibrium speed of the water droplets in ascending air.

8. The method as claimed in claim 7, wherein the ascending movement is ensured by suction in the upper part of the tube.

9. A device for measuring relative humidity and temperature of the air in a diphasic medium consisting of air and water droplets, in which a relative humidity sensor (1) and a temperature sensor (2) are connected to a measurer (3), and in which an ascending air flow, ensured by suction in the upper part of the tube (4), passes through a substantially vertical tube (4), the relative humidity sensor (1) and the temperature sensor (2) being placed inside the tube (4), wherein the speed of ascent of the air in the tube (4) is less than the equilibrium speed of the water droplets in ascending air.

10. The measuring device as claimed in claim 9, wherein the suction is ensured by means of a pump.

11. The measuring device as claimed in claim 9, wherein the average distance between the lower opening of the tube (4) and the relative humidity (1) and temperature (2) sensors is about two times the diameter of the tube (4).
